(19) United States
(12) Patent Application Publication
(10) Pub. No.: US 2013/0144182 A1
(43) Pub. Date: Jun. 6, 2013

(54) SLEEP RESPIRATORY DISORDER EXAMINATION APPARATUS AND METHOD THEREOF

(71) Applicants: Ji Wook JEONG, Daejeon (KR); YongWon Jang, Daejeon (KR); Hyung Wook Noh, Daejeon (KR); Sooyeul Lee, Daejeon (KR); Seunghwan Kim, Daejeon (KR)

(72) Inventors: Ji Wook JEONG, Daejeon (KR); YongWon Jang, Daejeon (KR); Hyung Wook Noh, Daejeon (KR); Sooyeul Lee, Daejeon (KR); Seunghwan Kim, Daejeon (KR)

(73) Assignee: Electronics and Telecommunications Research Institute, Daejeon (KR)

(21) Appl. No.: 13/560,601

(22) Filed: Jul. 27, 2012

(30) Foreign Application Priority Data
Dec. 6, 2011 (KR) 10-2011-0129845

(51) Int. Cl. A61B 5/113

(52) U.S. Cl. 600/534

(57) ABSTRACT

Disclosed is a sleep respiratory disorder examining device which includes a measurement unit which includes at least one conductive fiber and measures a resistance variation in the conductive fiber, a length of the conductive fiber being varied according to movement of a user; and a signal processor which analyzes a sleeping state of the user based on a resistance variation measured by the measurement unit.

### CROSS-REFERENCE TO RELATED APPLICATIONS

A claim for priority under 35 U.S.C. §119 is made to Korean Patent Application No. 10-2011-0129845 filed Dec. 6, 2011, in the Korean Intellectual Property Office, the entire contents of which are hereby incorporated by reference.

### BACKGROUND

The inventive concepts described herein relate to a sleep respiratory disorder examining apparatus and a method thereof.

A sleeping state examining technique may be needed to make a deep sleep condition and to diagnose abnormal states such apnea, snoring, and the like. A sleeping state may be judged by detecting bio-signals (e.g., breathing, brainwave, electrocardiogram, eye movement, body turning, and the like) and estimating a sleeping level. A variation in breathing may be measured by collecting inspiration and exhalation and electrically measuring a flow of the air passing through a filter.

However, the above-described method may necessitate an external separate apparatus, and a measuring apparatus may be attached to a mouth of a person to be measured. This may hinder a deep sleep of a person to be measured. As a result, it is difficult to perform exact measurement.

### SUMMARY

Example embodiments of the inventive concept provide a sleep respiratory disorder examining device which comprises a measurement unit which includes at least one conductive fiber and measures a resistance variation in the conductive fiber, a length of the conductive fiber being varied according to movement of a user; and a signal processor which analyzes a sleeping state of the user based on a resistance variation measured by the measurement unit.

In example embodiments, the sleep respiratory disorder examining device further comprises a display unit which displays a sleeping state analyzed by the signal processor.

In example embodiments, the display unit includes a liquid crystal screen which displays a sleeping state analyzed by the signal processor in real time.

In example embodiments, the display unit includes a speaker which generates an alarm indicating an abnormal state analyzed by the signal processor in real time.

In example embodiments, the sleep respiratory disorder examining device further comprises a transmitter which sends a sleeping state analyzed by the signal processor to an external device.

In example embodiments, the signal processor comprises a voltage converting unit which converts a resistance variation measured by the measurement unit into a voltage; an analog-to-digital converting unit which converts a voltage measured by the voltage converting unit into a digital value; and a signal computing unit which analyzes a sleeping state of the user based on a digital value converted by the analog-to-digital converting unit.

In example embodiments, the voltage converting unit includes at least one voltage divider, the voltage divider including a reference resistor and a variable resistor.

In example embodiments, the analog-to-digital converting unit includes at least one analog-to-digital converting module and digitizes a voltage measured by the voltage converting unit into a multi-bit digital value through the at least one analog-to-digital converting module.

In example embodiments, the signal computing unit filters a digital value converted by the analog-to-digital converting unit to extract a breathing signal band.

In example embodiments, a sleeping state analyzed by the signal computing unit includes a variation in a breathing rate.

In example embodiments, a sleeping state analyzed by the signal computing unit includes an apnea state.

In example embodiments, a sleeping state analyzed by the signal computing unit includes a periodic limb movement disorder.

In example embodiments, the measurement unit is inserted in a matrix.

In example embodiments, the measurement unit is inserted in a pillow.

In example embodiments, the measurement unit is attached to bedclothes.

In example embodiments, the measurement unit has a band shape to be attached to a user.

Example embodiments of the inventive concept provide a sleep respiratory disorder examining method comprising measuring a resistance variation in a conductive fiber due to movement of a body of a user; converting the measured resistance variation into a voltage; digitizing the voltage into a digital value; analyzing a bio-signal based on the digitized digital value; and displaying the analyzed bio-signal through a display unit and transmitting the analyzed bio-signal to an external device.

### BRIEF DESCRIPTION OF THE FIGURES

The above and other objects and features will become apparent from the following description with reference to the following figures, wherein like reference numerals refer to like parts throughout the various figures unless otherwise specified, and wherein

FIG. 1 is a block diagram schematically illustrating a sleep respiratory disorder examining device according to an embodiment of the inventive concept.

FIG. 2 is a detailed block diagram schematically illustrating a sleep respiratory disorder examining device in FIG. 1.

FIG. 3 is a block diagram schematically illustrating a sleep respiratory disorder examining device according to another embodiment of the inventive concept.

FIG. 4 is a block diagram schematically illustrating a sleep respiratory disorder examining device according to still another embodiment of the inventive concept.

FIG. 5 is a conceptual diagram illustrating a sleep respiratory disorder examining device according to an embodiment of the inventive concept.

FIG. 6 is a conceptual diagram illustrating a sleep respiratory disorder examining device according to another embodiment of the inventive concept.

FIG. 7 is a graph illustrating a resistance variation of a variable resistor according to time lapse.

FIG. 8 is a flowchart illustrating a sleep respiratory disorder examining method according to an embodiment of the inventive concept.

### DETAILED DESCRIPTION

Embodiments will be described in detail with reference to the accompanying drawings. The inventive concept, however, may be embodied in various different forms, and should not be construed as being limited only to the illustrated embodiments. Rather, these embodiments are provided as examples so that this disclosure will be thorough and complete, and will fully convey the concept of the inventive concept to those skilled in the art. Accordingly, known processes, elements, and techniques are not described with respect to some of the embodiments of the inventive concept. Unless otherwise noted, like reference numerals denote like elements throughout the attached drawings and written description, and thus descriptions will not be repeated. In the drawings, the sizes and relative sizes of layers and regions may be exaggerated for clarity.

It will be understood that, although the terms “first”, “second”, “third”, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the inventive concept.

Spatially relative terms, such as “beneath”, “below”, “lower”, “under”, “above”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features would then be oriented “above” the other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as being “between” two layers, it can be the only layer between the two layers, or one or more intervening layers may also be present.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Also, the term “exemplary” is intended to refer to an example or illustration.

It will be understood that when an element or layer is referred to as being “on”, “connected to”, “coupled to”, or “adjacent to” another element or layer, it can be directly on, connected, coupled, or adjacent to the other element or layer, or intervening elements or layers may be present. In contrast, when an element is referred to as being “directly on,” “directly connected to”, “directly coupled to”, or “immediately adjacent to” another element or layer, there are no intervening elements or layers present.

Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

FIG. 1 is a block diagram schematically illustrating a sleep respiratory disorder examining device according to an embodiment of the inventive concept. Referring to FIG. 1, a sleep respiratory disorder examining device 100 may include a measurement unit 110 and a signal processor 120.

The measurement unit 110 may measure movement of a chest at breathing. The measurement unit 110 may include at least one variable resistor. A value of the variable resistor in the measurement unit 110 may be varied according to movement of the chest at breathing. The variable resistor of the measurement unit 110 may be formed of a conductive fiber. A length of the conductive fiber of the measurement unit 110 may be varied according to movement of a body, (e.g., a chest) at breathing. Internal resistance of the conductive fiber may be varied according to a variation in length. Thus, a variation in the internal resistance of the conductive fiber may indicate movement of a chest at breathing of a user.

The conductive fiber may include a conductive thread and an elastic thread. With elasticity of the elastic thread, a length of the conductive fiber may be varied according to a variation of a body at breathing. The conductive thread may be formed of a carbon thread or a metal line. The conductive fiber may be formed to have a twisted shape of the conductive thread and the elastic thread. For example, the conductive fiber may have such a shape that the conductive thread wraps the elastic thread. Alternatively, the conductive fiber may have such a shape that the conductive thread and the elastic thread are twisted in opposite directions. However, the inventive concept is not limited thereto.

The measurement unit 110 may be embedded within a matrix or a pillow that contacts with a body of a user. The sleep respiratory disorder examining device 100 including the measurement unit 110 may be provided as a new item such as the dedicated bedclothes. Alternatively, the measurement unit 110 may be additionally attached to an existing sleeping product. Alternatively, the measurement unit 110 may be attached to a jacket in a band shape, or may be inserted in a clothing of a user. The measurement unit 110 can be attached to a body of a user in a belt shape. With the above description, the measurement unit 110 may measure a breathing state through movement of a body without restricting of a user. The measurement unit 110 may send the measured resistance variation in the variable resistor to the signal processor 120.

The signal processor 120 may analyze a breathing state of a user based on a resistance value provided from the measurement unit 110. For example, the signal processor 120 may calculate a variation in a breathing rate of the user. Alternatively, the signal processor 120 may judge whether the user is at an apnea state. However, the inventive concept is not limited thereto.

The sleep respiratory disorder examining device 100 in FIG. 1 may examine a sleeping state without an external complicated apparatus as it does not hinder a sleep of the user.

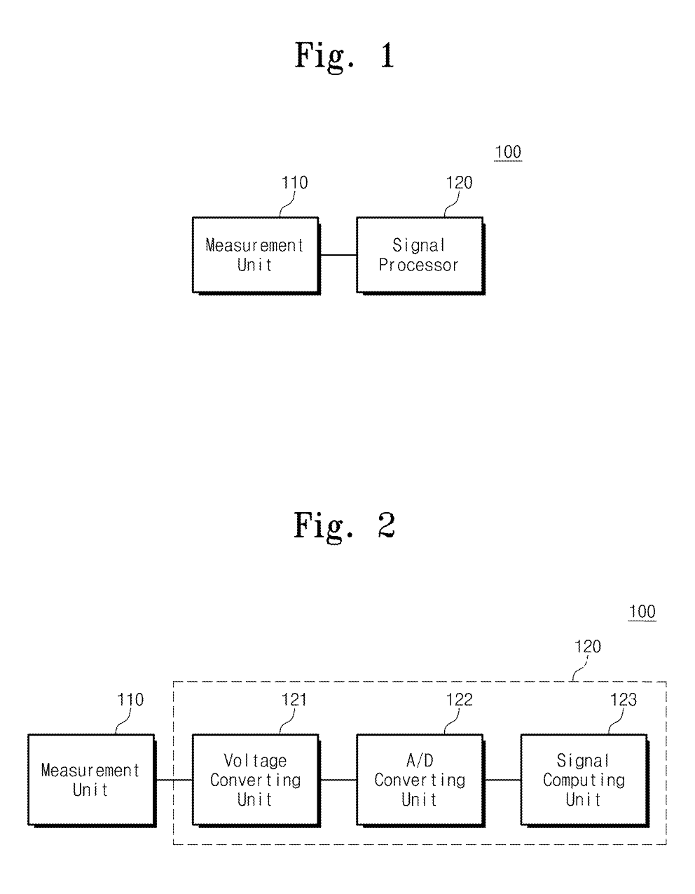

FIG. 2 is a detailed block diagram schematically illustrating a sleep respiratory disorder examining device in FIG. 1. Referring to FIG. 2, a signal processor 120 may include a voltage converting unit 121, an analog-to-digital (A/D) converting unit 122, and a signal computing unit 123.

The voltage converting unit 121 may convert a resistance value input from a measurement unit 1110 into a voltage. In example embodiments, the voltage converting unit 121 may include one or more voltage dividers. A voltage divider may include a reference resistor and a variable resistor connected in series between a power terminal and a ground terminal. The variable resistor may indicate a resistance value provided from the measurement unit 110. A voltage across the variable resistor may be varied when an input resistance value is varied according to movement of a body (e.g., a chest) at breathing. The voltage converting unit 121 may convert a resistance value into a voltage by measuring the voltage.

The A/D converting unit 122 may convert an analog voltage measured by the voltage converting unit 121 into a digital value. The A/D converting unit 122 may include at least one A/D converting module. The A/D converting unit 122 may digitize a voltage into a multi-bit digital value through A/D converting modules.

The signal computing unit 123 may analyze a sleeping state of a user based on a digital value digitized by the A/D converting unit 122. In general, an adult may toss and turn 20 to 30 times during a sleeping time. When measured according to movement of a body, an error may be generated due to such breaths. The error may be removed by processing a signal using a filter.

For example, the signal computing unit 123 may filter the digital value to extract a breathing signal band. The signal computing unit 123 may analyze the extracted value to calculate variations in breathing strength and rate. The signal computing unit 123 may diagnose whether a user is at an abnormal state during sleeping, based on the calculated value. For example, the signal computing unit 123 may diagnose an apnea state of a user based on a variation in a breathing rate. Also, the signal computing unit 123 may diagnose a periodic limb movement disorder (PLMD) of the user. The periodic limb movement disorder may be a sleep disorder where the user moves limbs involuntarily during sleep.

As described above, a sleeping state of a user may be examined by measuring movement of a body (e.g., a chest) of the user using a variable resistor and digitizing the measured value. The sleep respiratory disorder examining device 100 can be configured such that a user or an observer checks a diagnosed result in real time, which will be more fully described later.

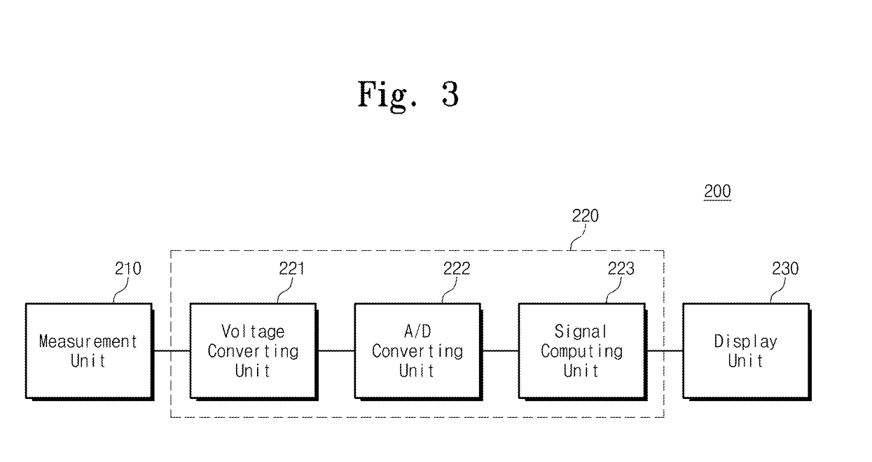

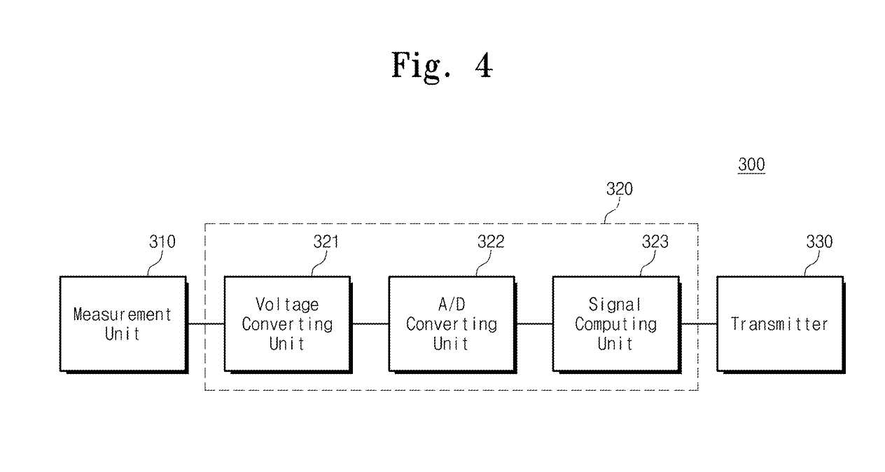

FIG. 3 is a block diagram schematically illustrating a sleep respiratory disorder examining device according to another embodiment of the inventive concept. A sleep respiratory disorder examining device 200 in FIG. 3 may be configured the same as that in FIG. 2 except that a display unit 230 is added. Referring to FIG. 3, a sleep respiratory disorder examining device 200 may include a measurement unit 210, a signal processor 220, and a display unit 230.

The display unit 230 may display a sleeping state analyzed by the signal processor 220. The display unit 230 may include an image device and an audio device. For example, the display unit 230 may include a liquid crystal display or a speaker. In example embodiments, the display unit 230 may show a measured result of a breathing rate through the liquid crystal screen in real time. Also, the display unit 230 may generate an alarm informing an abnormal state (e.g., apnea) through a speaker.

The sleep respiratory disorder examining device 200 may examine a sleeping state of a user, and may output the examined result through an output device (e.g., an image device or an audio device) such that the user or an observer checks the examined result in real time. Thus, it is possible to immediately cope with an abnormal state of the user.

A sleep respiratory disorder examining device in FIG. 2 can be configured such that a diagnosed result is analyzed more precisely at an external device, which will be more fully described later.

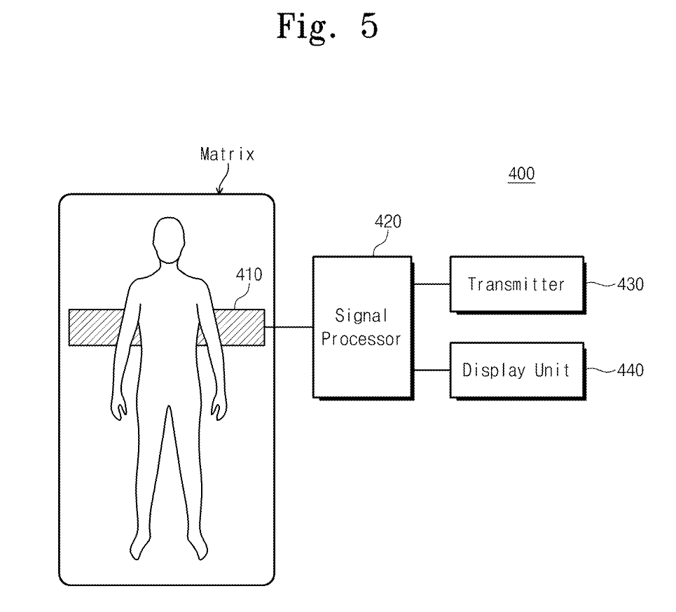

FIG. 4 is a block diagram schematically illustrating a sleep respiratory disorder examining device according to still another embodiment of the inventive concept. A sleep respiratory disorder examining device 300 in FIG. 4 may be configured the same as that in FIG. 2 except that a transmitter 330 is added. Referring to FIG. 4, a sleep respiratory disorder examining device 300 may include a measurement unit 310, a signal processor 320, and a transmitter 330.

The transmitter 230 may transmit a sleeping state analyzed by the signal processor 220 to an external device. The transmitter 230 may transmit the sleeping state in a wire or wireless manner. The sleeping state transmitted from the transmitter 330 may be additionally analyzed by an external module.

The sleep respiratory disorder examining device 300 in FIG. 4 may examine a sleeping state of a user, and may transmit the examined result to an external device for more precise analysis. Thus, the sleep respiratory disorder examining device 300 in FIG. 4 may provide a precise analysis result compared with a sleep respiratory disorder examining device in FIG. 2.

FIG. 5 is a conceptual diagram illustrating a sleep respiratory disorder examining device according to an embodiment of the inventive concept. Referring to FIG. 5, a sleep respiratory disorder examining device 400 may include a measurement unit 410, a signal processor 420, a transmitter 430, and a display unit 440. A sleep respiratory disorder examining device 400 in FIG. 5 may be identical to that in FIG. 3 except that a display unit 440 is added.

In example embodiments, the measurement unit 410 may be inserted in a bed matrix. The measurement unit 410 may be configured to be extended in a horizontal direction. Alternatively, the measurement unit 410 may be configured to be extended in a vertical direction. The measurement unit 410 may include one or more variable resistors, which are disposed in parallel to be spaced apart from one another. Alternatively, variable resistors in the measurement unit 410 may be disposed to be paired. Alternatively, variable resistors in the measurement unit 410 may be disposed to form a matrix form. The measurement unit 410 may measure resistance values varied at one or more places. The sleep respiratory disorder examining device 400 may reduce an error due to movement not associated with a measurement environment (e.g., a breath of a user). However, the inventive concept is not limited thereto.

A body of a user, for example, a chest may move according to a breathing of the user. Resistance of a variable resistor of the measurement unit 410 may be varied according to movement of a chest of the user contacting with a surface of a matrix. The sleep respiratory disorder examining device 400 may analyze a sleeping state of the user using the varied resistance value.

The sleep respiratory disorder examining device 400 may provide sleeping state information of a user in real time without inconvenience of the user by inserting a measurement unit 410 in a bed. Thus, it is possible to obtain a more precise result by measuring a sleeping state at a deep sleep state without hindering.

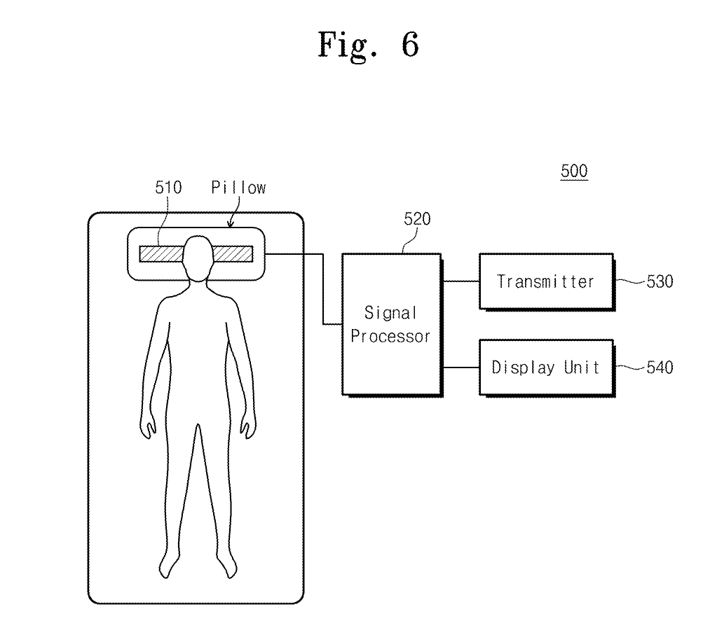

FIG. 6 is a conceptual diagram illustrating a sleep respiratory disorder examining device according to another embodiment of the inventive concept. Referring to FIG. 6, a sleep respiratory disorder examining device 500 may include a measurement unit 510, a signal processor 520, a transmitter 530, and a display unit 540. A sleep respiratory disorder examining device 400 in FIG. 6 may be analogous to that in FIG. 5.

In example embodiments, the measurement unit 510 may be inserted in a pillow. The measurement unit 510 may be configured to be extended in a horizontal direction. The measurement unit 510 may include one or more variable resistors, which are disposed in parallel to be spaced apart from one another. Alternatively, variable resistors in the measurement unit 410 may be disposed to be paired. However, the inventive concept is not limited thereto.

A body of a user (e.g., a head) may move according to a breathing of the user. Resistance of a variable resistor of the measurement unit 510 may be varied according to movement of a head of the user contacting with a surface of a pillow. The sleep respiratory disorder examining device 500 may analyze a sleeping state of the user using the varied resistance value.

The sleep respiratory disorder examining device 500 may provide sleeping state information of a user in real time without inconvenience of the user by inserting a measurement unit 510 in a pillow. Thus, it is possible to obtain a more precise result by measuring a sleeping state at a deep sleep state without hindering.

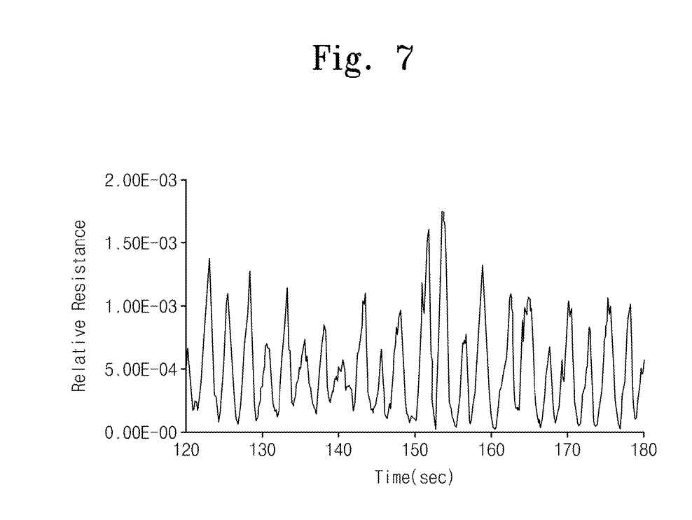

FIG. 7 is a graph illustrating a resistance variation of a variable resistor according to time lapse. There may be understood from FIG. 7 that a resistance value of a variable resistor periodically varies according to time lapse. A variation in the number of peak values of the variable resistor may show a variation in a breathing rate. That is, it is possible to estimate a breathing rate using a variation of a resistance value.

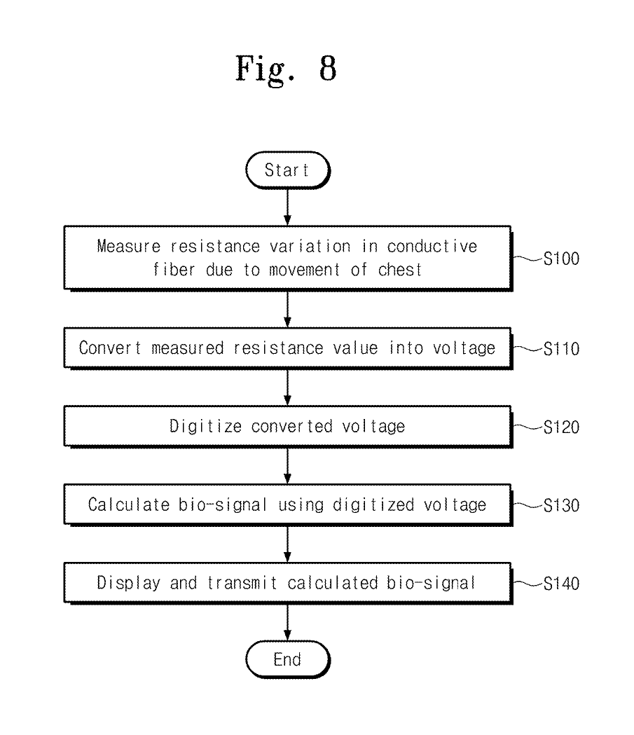

FIG. 8 is a flowchart illustrating a sleep respiratory disorder examining method according to an embodiment of the inventive concept. Referring to FIG. 8, in operation S110, a variation in resistance of a conductive fiber indicating movement of a body of a user may be measured. In operation S110, the measured resistance value may be converted into an analog voltage. In operation S120, the converted voltage may be digitized into a digital value. In operation S130, a bio-signal (e.g., a breathing rate or an abnormal state during sleeping) may be analyzed according to a voltage digitized into a multi-bit digital value. In operation S140, the analyzed bio-signal may be displayed through a display unit such as a liquid crystal or an audio device, or may be output to an external device by wire or wireless.

The inventive concept may be modified or changed variously. For example, a measurement unit, a signal processor, a display unit, and a transmitter may be changed or modified variously according to environment and use.

While the inventive concept has been described with reference to exemplary embodiments, it will be apparent to those skilled in the art that various changes and modifications may be made without departing from the spirit and scope of the present invention. Therefore, it should be understood that the above embodiments are not limiting, but illustrative.

What is claimed is:

1. A sleep respiratory disorder examining device comprising:
a measurement unit which includes at least one conductive fiber and measures a resistance variation in the conductive fiber, a length of the conductive fiber being varied according to movement of a user; and
a signal processor which analyzes a sleeping state of the user based on the resistance variation measured by the measurement unit.

2. The sleep respiratory disorder examining device of claim 1, further comprising:
a display unit which displays the sleeping state analyzed by the signal processor.

3. The sleep respiratory disorder examining device of claim 2, wherein the display unit includes a liquid crystal screen which displays the sleeping state analyzed by the signal processor in real time.

4. The sleep respiratory disorder examining device of claim 2, wherein the display unit includes a speaker which generates an alarm indicating an abnormal state analyzed by the signal processor in real time.

5. The sleep respiratory disorder examining device of claim 1, further comprising:
a transmitter which sends the sleeping state analyzed by the signal processor to an external device.

6. The sleep respiratory disorder examining device of claim 1, wherein the signal processor comprises:
a voltage converting unit which converts a resistance variation measured by the measurement unit into a voltage;
an analog-to-digital converting unit which converts a voltage measured by the voltage converting unit into a digital value; and
a signal computing unit which analyzes a sleeping state of the user based on a digital value converted by the analog-to-digital converting unit.

7. The sleep respiratory disorder examining device of claim 6, wherein the voltage converting unit includes at least one voltage divider, the voltage divider including a reference resistor and a variable resistor.

8. The sleep respiratory disorder examining device of claim 6, wherein the analog-to-digital converting unit includes at least one analog-to-digital converting module and digitizes a voltage measured by the voltage converting unit into a multi-bit digital value through the at least one analog-to-digital converting module.

9. The sleep respiratory disorder examining device of claim 6, wherein the signal computing unit filters a digital value converted by the analog-to-digital converting unit to extract a breathing signal band.

10. The sleep respiratory disorder examining device of claim 6, wherein a sleeping state analyzed by the signal computing unit includes a variation in a breathing rate.

11. The sleep respiratory disorder examining device of claim 6, wherein a sleeping state analyzed by the signal computing unit includes an apnea state.

12. The sleep respiratory disorder examining device of claim 6, wherein a sleeping state analyzed by the signal computing unit includes a periodic limb movement disorder.

13. The sleep respiratory disorder examining device of claim 1, wherein the measurement unit is inserted in a matrix.

14. The sleep respiratory disorder examining device of claim 1, wherein the measurement unit is inserted in a pillow.

15. The sleep respiratory disorder examining device of claim 1, wherein the measurement unit is attached to bedclothes.

16. The sleep respiratory disorder examining device of claim 1, wherein the measurement unit has a band shape to be attached to a user.

17. A sleep respiratory disorder examining method comprising:
measuring a resistance variation in a conductive fiber due to movement of a body of a user;
converting the measured resistance variation into a voltage;
digitizing the voltage into a digital value;
analyzing a bio-signal based on the digitized digital value; and
displaying the analyzed bio-signal through a display unit and transmitting the analyzed bio-signal to an external device.
